(19) United States
(12) Patent Application Publication
(10) Pub. No.: US 2020/0332249 A1
(43) Pub. Date: Oct. 22, 2020

(54) PRODUCTION OF IRON-COMPLEXED PROTEINS FROM ALGAE

(71) Applicant: Triton Algae Innovations, Inc., San Diego, CA (US)

(72) Inventors: Miller Tran, San Diego, CA (US); Brock Adams, San Diego, CA (US); John Deaton, San Diego, CA (US); Oscar Gonzalez, San Diego, CA (US); Jon Hansen, San Diego, CA (US); Amanda Longo, San Diego, CA (US); Michael Mayfield, San Diego, CA (US); Xun Wang, San Diego, CA (US)

(21) Appl. No.: 16/850,842

(22) Filed: Apr. 16, 2020

Related U.S. Application Data
(60) Provisional application No. 62/835,761, filed on Apr. 18, 2019.

(51) Int. Cl. C12N 1/12; A23J 3/20; A23L 33/105

(52) U.S. Cl. CPC C12N 1/12; C12R 1/89; A23L 33/105; A23J 3/20

(57) ABSTRACT

Provided herein are recombinant microalgae containing one or more polynucleotides encoding iron-complexed proteins, methods of producing the iron-complexed proteins with the microalgae, and edible products formed therefrom.

### CROSS REFERENCE TO RELATED APPLICATION(S)

This application claims the benefit of priority under 35 U.S.C. § 119(e) of U.S. Ser. No. 62/835,761, filed Apr. 18, 2019, the entire content of which is incorporated herein by reference.

### SEQUENCE LISTING

The instant application contains a Sequence Listing which has been submitted electronically in ASCII format and is hereby incorporated by reference in its entirety. Said ASCII copy, created on Apr. 16, 2020, is named 20498-102512_SL.txt and is 27 kilobytes in size.

### BACKGROUND

World populations continue to grow and this growth has resulted in an increase in the amount of animal meat that is consumed. However, the consumption of animal meat has many negative consequences that range from environmental to moral. Livestock animals are fed plants which they ingest to provide energy and also which they convert into their own body mass. This inefficient conversion results in a large amount of waste, including methane, carbon dioxide, agricultural waste and unusable animal material, as only a small portion of the plant is converted into animal meat. For example, beef production requires 20 times more land than beans, peas, and lentils to produce the same amount of protein, and emits 20 times more greenhouse gases. Additionally, the demand for meat has led to the industrial production of animals which raises ethical concerns for animal growth conditions and animal cruelty.

To decrease the demand of animal meat there is an effort by the food industry to provide alternatives to animal meat through the production of plant-based meat substitutes. However, these options are often times lacking in flavor, meat like color, aroma, mouth-feel, texture and others. One strategy that has been developed to mimic the bleeding of meat that occurs when it is cooked is to add an iron-complexed protein such as leghemoglobin to plant-based meat products. Leghemoglobin is a plant-based iron-complexed protein whose biochemical properties mimic those of myoglobin, an animal iron-complexed protein. When added to plant-based meat substitutes both leghemoglobin and myoglobin cause the meat to have a reddish pink appearance and results in a plant-based meat that bleeds when it cooks. Additionally, the ability of both leghemoglobin and myoglobin to bind to heme co-factors results in plant-based meat substitutes that have flavors similar to that of animal meat than a plant.

Currently, there are limited vegan methods of production of such iron-complexed proteins including isolation from natural plant sources such as from root nodules and recombinant production in micro-organisms such as the metholytrophic yeast, Pichia pastoris. However, Pichia is not an organism that is generally regarded as safe to consume, making it necessary to isolate the iron-complexed protein from the yeast. The cost of purification drastically increases the cost to produce plant-based meat alternatives. Accordingly, there is a need for new methods and approaches for producing iron-complexed proteins to improve the cost of plant-based meat alternatives and other foodstuffs and nutritional supplements containing iron.

### SUMMARY OF THE INVENTION

Provided herein are methods and compositions for the recombinant production of iron-complexed proteins in microalgae that can then be incorporated into foodstuffs and nutritional supplements, including plant-based meat alternatives either as a part of whole cell algae or as a purified protein.

In various aspects, the invention provides a recombinant microalgae that includes a nucleic acid molecule encoding a heterologous iron-complexed protein and the microalgae is capable of accumulating the iron-complexed protein to at least about 0.1% of the algae biomass. In various embodiments, the microalgae is a Chlamydomonas Sp, such as Chlamydomonas reinhardtii. In various embodiments, the heterologous iron-complexed protein is selected from hemoglobin, myoglobin, leghemoglobin, beta hemoglobin, alpha hemoglobin, flavorhemoglobin, histoglobin, a neuroglobin, a protoglobin, truncated globin, and lactoferrin. The iron-complexed proteins useful in the compositions and methods herein include hemoglobin, myoglobin, leghemoglobin, beta hemoglobin, alpha hemoglobin, flavohemoglobin, histoglobin, a neuroglobin, a protoglobin, truncated globin, and lactoferrin. In some embodiments, the nucleic acid molecule encoding the iron-complexed protein is regulated by a heterologous promoter. The nucleic acid molecule encoding the iron-complexed protein may be integrated into the nuclear genome and/or the chloroplast genome of the microalgae. In some embodiments, the nucleic acid is integrated into the mitochondrial genome of the microalgae.

In some embodiments, the nucleic acid molecule encoding the iron-complexed protein has a nucleic acid sequence of any of SEQ ID NOs: 1 or 2, or a sequence with at least 80% identity thereto. In some embodiments, the nucleic acid molecule encoding the iron-complexed protein is codon-optimized for expression in the microalgae. The microalgae useful for the compositions and methods herein also may include one or more additional nucleic acid molecules encoding a protein to enhance the accumulation or stability of the iron-complexed protein.

In various aspects, the invention also provides a composition that includes a microalgae biomass or a portion thereof as described herein. In various embodiments, the heterologous iron-complexed protein included in such compositions is selected from hemoglobin, myoglobin, leghemoglobin, beta hemoglobin, alpha hemoglobin, flavorhemoglobin, histoglobin, a neuroglobin, a protoglobin, truncated globin, and lactoferrin. In various embodiments, the composition comprises the whole microalgae biomass.

The microalgae compositions and methods provided herein may be used to create human and/or animal consumable substances including a foodstuff, a nutritional supplement, a plant-based meat substitute, an imitation seafood, an imitation meat and/or a neutraceutical. Accordingly, in various aspects, the invention also provides an edible product that includes the composition provided herein. In various embodiments, the composition includes a portion of the microalgae biomass that is enriched in the heterologous iron-complexed protein as compared to the whole microalgae biomass. In various embodiments, the edible product is selected from the group consisting of a beverage, a food, a food supplement, a nutraceutical, an imitation meat and an imitation seafood.

In various aspects, the invention also provides methods of making an edible product. The methods include obtaining a biomass from the recombinant microalgae as disclosed herein and combining the biomass or a portion thereof containing the heterologous iron-complexed protein with at least one edible ingredient to create an edible product. In various embodiments, the edible product is selected from the group consisting of a beverage, a food, a food supplement, a nutraceutical, an imitation meat and an imitation seafood. In various embodiments, the heterologous iron-complexed protein is isolated from the biomass prior to incorporating the iron-complexed protein into the edible product.

In various aspects, the invention also provides methods for producing a recombinant iron-complexed protein in algae. The methods, which include the steps of integrating a nucleic acid molecule encoding an iron-complexed protein into a microalgae genome, growing the microalgae under conditions sufficient to express the iron-complexed protein in its biomass; and harvesting the microalgae biomass to recover the produced iron-complexed protein. In various embodiments, the iron-complexed protein is hemoglobin, myoglobin, leghemoglobin, beta hemoglobin, alpha hemoglobin, flavorhemoglobin, histoglobin, a neuroglobin, a protoglobin, truncated globin, or lactoferrin. In various embodiments, the method also includes growing the microalgae heterotrophically or mixotrophically using a reduced carbon source selected from the group consisting of fructose, sucrose, glucose, galactose, maltose, acetate and an organic acid. In various embodiments, the microalgae is a Chlamydomonas sp. In various embodiments, the method also includes incorporating the produced iron-complexed protein into an edible product, wherein the edible product is selected from the group consisting of beverage, a food, a food supplement, a nutraceutical, an imitation meat and an imitation seafood. In various embodiments, the method also includes separating the iron-complexed protein from the microalgae biomass prior to incorporating the produced iron-complexed protein into the edible product. In some embodiments, the iron-complexed protein is at least 0.1% of the harvested algae biomass. In some embodiments, the nucleic acid is integrated into the microalgal nuclear genome, and/or the microalgal chloroplast genome and/or the microalgal mitochondrial genome.

In any of the methods provided herein, the microalgae may be grown heterotrophically or mixotrophically using a reduced carbon source. In some embodiments, the algae is cultivated in a bioreactor which is supplied with air or another gas mixture rich in oxygen. In some embodiments, the algae density reaches at least 100 g/l. In some embodiments, the algae density reaches at least about 100 g/l in less than 7 days. In some embodiments, the dissolved oxygen content in the bioreactor is allowed to drop below 5% once the iron-carrying protein concentration exceeds 0.1% of the algal biomass. In some embodiments, the microalgae is grown under aerobic fermentation conditions.

In some embodiments, the algae used to express the iron-complexed protein turns yellow in the dark. In some embodiments, the algae used to express the iron-complexed protein turns white in the dark. In some embodiments, the algae strain is one with a reduced chlorophyll content. In some embodiments, the algae accumulates heme co-factors at least 0.1% of biomass. In some embodiments, the reduced the carbon source for the growth of the algae is a sugar. In some embodiments, the sugar is fructose, sucrose, glucose, galactose, or maltose. In some embodiments, the selected reduced carbon source is not an alcohol. In some embodiments, the reduced carbon source is an acetate or an organic acid. In some embodiments, the algae is grown in the dark.

### BRIEF DESCRIPTION OF THE DRAWINGS

FIG. 1 is a pictorial diagram showing a western blot of the green algae, Chlamydomonas reinhardtii, expressing myoglobin in its chloroplasts. The expression of myoglobin is increased in the dark when compared to the light. The western blot was probed with an antibody against cow myoglobin.

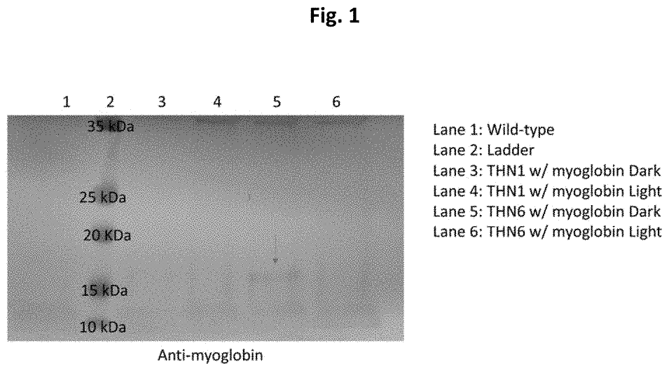

### DETAILED DESCRIPTION

Provided herein are methods and compositions for the recombinant production of iron-complexed proteins, such as leghemoglobin and myoglobin, in algae that can then be incorporated into foodstuffs and nutritional supplements, including plant-based meat alternatives, either as a part of whole cell algae or as a purified protein. Production and/or delivery of iron-complexed proteins in edible microalgae finds use, e.g., in mammalian health and nutrition. These uses include the ability of these proteins to impart meat-like flavoring and/or meat-like coloring to edible products. This can be done using methods known in the art to produce products that are vegan but have meat-like characteristics.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

The term “comprising,” which is used interchangeably with “including,” “containing,” or “characterized by,” is inclusive or open-ended language and does not exclude additional, unrecited elements or method steps. The phrase “consisting of” excludes any element, step, or ingredient not specified in the claim. The phrase “consisting essentially of” limits the scope of a claim to the specified materials or steps and those that do not materially affect the basic and novel characteristics of the claimed invention. The present disclosure contemplates embodiments of the invention compositions and methods corresponding to the scope of each of these phrases. Thus, a composition or method comprising recited elements or steps contemplates particular embodiments in which the composition or method consists essentially of or consists of those elements or steps.

As used herein, the term “gene” means the deoxyribonucleotide sequences comprising the coding region of a structural gene. A “gene” may also include non-translated sequences located adjacent to the coding region on both the 5′ and 3′ ends such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene which are transcribed into heterogenous nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, a “regulatory gene” or “regulatory sequence” is a nucleic acid sequence that encodes products (e.g., transcription factors) that control the expression of other genes.

As used herein, a “protein coding sequence” or a sequence that encodes a particular protein or polypeptide, is a nucleic acid sequence that is transcribed into mRNA (in the case of DNA) and is translated (in the case of mRNA) into a polypeptide in vitro or in vivo when placed under the control of appropriate regulatory sequences. The boundaries of the coding sequence are determined by a start codon at the 5′ terminus (N-terminus) and a translation stop nonsense codon at the 3′ terminus (C-terminus). A coding sequence can include, but is not limited to, cDNA from eukaryotic mRNA, genomic DNA sequences from eukaryotic DNA, and synthetic nucleic acids. A transcription termination sequence will usually be located 3′ to the coding sequence.

As used herein, “phototrophic” growth or growing conditions refers to conditions where an organism (e.g., a microalgae) uses photon capture as a source of energy and can fix inorganic carbon. As such phototrophic organisms, such as phototrophic microalgae, are capable of using inorganic carbon in the presence of light as a source of metabolic carbon.

As used herein, “heterotrophic” growth or growing conditions refers to conditions where an organism (e.g., a microalgae) does not use photon capture as an energy source, but instead relies on organic carbon sources.

As used herein, “mixotrophic” growth or growing conditions refers to conditions where an organism (e.g., a microalgae) is capable of using photon capture and inorganic carbon fixation to support growth, but in the absence of light may use organic carbon as an energy source. Thus, mixotrophic growth has the metabolic characteristics of both phototrophic and heterotrophic conditions.

Iron-complexed proteins are synthesized in algae by using polypeptide expression techniques. To do so, a synthetic gene that codes for the amino acid of an iron-complexed protein is synthesized and ligated into a DNA transformation vector that facilitates the integration of the exogenous gene into the algal genome. In some embodiments, the gene coding for the iron-complexed protein is integrated into the nuclear genome. In some embodiments the gene coding for the iron-complexed protein is integrated into the chloroplast genome. In some embodiments the gene coding for the iron-complexed protein is integrated into the mitochondrial genome. In some embodiments the gene encoding for the iron-complexed protein has a nucleic acid sequence of a naturally occurring gene from an animal, microbial or plant species. In some embodiments, the nucleic acid molecule encoding iron-complexed protein is codon optimized to maximize expression in the nuclear, chloroplast or mitochondrial genome of algae.

In some embodiments, a gene encoding an iron-complexed protein may have altered codons for expression from the nuclear or chloroplast genomes of edible microalgae, including Chlamydomonas reinhardtii. Illustrative iron-complexed proteins include without limitation myoglobin, leghemoglobin, hemoglobin, beta hemoglobin, alpha hemoglobin, flavohemoglobin, histoglobin, a neuroglobin, a protoglobin, truncated globin, and lactoferrin. The genes encoding for the iron-complexed protein can be integrated into and expressed from nuclear or chloroplast genomes of microalgae. Expression and bioactivity can be confirmed using methods known in the art.

Described herein are processes to produce iron-complexed proteins for health and nutrition purposes using microalgae. Thus, the organisms and processes described herein enable the large-scale production of iron-complexed proteins to be incorporated into food, health and nutrition products. Once strains of algae are produced that have the gene of an exogenous iron-complexed protein integrated into their genome, the algae can be grown in various conditions to improve the expression of the protein. In various embodiments, the iron-complexed protein is grown in autorophic conditions in the light. In various embodiments the iron-complexed protein is grown in mixotrophic conditions with a reduced carbon source and light. In various embodiments the iron-complexed protein is grown in heterotrophic conditions with a reduced carbon source and in the dark.

The presence of heme-cofactors can be integrated into the iron-complexed protein, such as a protoporphyrin. In various embodiments the iron-complexed protein can use the naturally occurring heme as a co-factor. In various embodiments the iron-complexed protein uses a heterologously produced heme co-factor.

A major advantage of algae is that many strains are Generally Regarded As Safe (GRAS) and can be used as food ingredients. This removes the need to purify the iron-complexed protein from the algal cellular material. In some embodiments, the iron-complexed protein is expressed and isolated in the chloroplasts. In some embodiments, the iron-complexed protein is expressed and isolated in the mitochondria. In some embodiments, the iron-complexed protein is expressed in the nucleus and isolated in the nuclear envelope. In some embodiments, the iron-complexed protein is expressed in the nucleus and sequestered to the endoplasmic reticulum. In some embodiments, the iron-complexed protein is expressed and isolated in the periplasmic space. In some embodiments, the iron-complexed protein is expressed in and resides in the chloroplast.

When the iron-complexed protein is sequestered inside the cell, the algal culture can be harvested and incorporated into plant-based meat substitutes to confer to the meat-based substitute with the properties of the iron-complexed protein resulting in a product that has attributes similar to animal meat.

In various embodiments, the algae used to express the iron-complexed protein can be a green, red, brown or golden microalgae.

In some embodiments, the strain of algae expressing the iron-complexed protein is grown in stainless steel fermentation vessels using batch-culture techniques. In some embodiments, the strain of algae expressing an iron-complexed protein is grown in stainless steel fermentation vessels using fed-batch culture techniques. In some embodiments, the strain of algae expressing the iron-complexed protein is grown in glass photobioreactors using a batch technique. In some embodiments, the strain of algae expressing the iron-complexed protein is grown in glass photobioreactors using a fed batch technique.

To increase the amount of stable iron-complexed protein that can be produced by the cell it may be beneficial to increase the amount of heme co-factor that is present. To increase the content of heme-cofactor, additional exogeneous genes are expressed that facilitate the increased production of the heme co-factor by the algal cell, including without limitation, one or more of ALA synthase, ALA dehydratase, porphobilinogen deaminase, UPG III synthase, UPG III decarboxylase, CPG oxidase, PPG oxidase, and ferrochelatase. Additionally, the expression of endogenous algae genes can be increased to facilitate the greater accumulation of heme-cofactor. The increase in heme-cofactor results in a greater accumulation of the heme-containing protein.

Hemoglobin proteins typically have a high affinity for oxygen. These proteins can therefore buffer the concentration of free oxygen inside plant cells. In a bioreactor for producing hemoglobin in micro-algae this can lead to a reduced dissolve oxygen requirement for a given oxygen transfer rate or a higher oxygen transfer rate at a given dissolved oxygen content.

### Polynucleotides Encoding Iron-Complexed Polypeptides and Their Expression

Microalgae described herein have genes encoding iron-complexed proteins integrated into the nuclear and/or chloroplast genome. In various embodiments, 1, 2, 3, 4, 5, 6, 7, 8, 9, 10, or more, polynucleotides encoding proteins used to increase the accumulation of the iron-complexed protein are independently integrated into the nuclear and/or chloroplast genome of microalgae.

Included as exemplary iron-complexed proteins are myoglobin, leghemoglobin, hemoglobin, beta hemoglobin, alpha hemoglobin, flavohemoglobin, histoglobin, a neuroglobin, a protoglobin, truncated globin, and lactoferrin. Proteins used to boost their expression include without limitation, ALA synthase, ALA dehydratase, porphobilinogen deaminase, UPG III synthase, UPG III decarboxylase, CPG oxidase, PPG oxidase, and ferrochelatase. In various embodiments, the iron-complexed protein is human, non-human primate, bovinae (e.g., cow, bison), ovine, caprine, camelid, canine, feline, equine, marsupial, or from any other mammal of interest.

The terms “polynucleotide” and “nucleic acid molecule” refer to single- or double-stranded polymers of deoxyribonucleotide or ribonucleotide bases read from the 5′ to the 3′ end. Polynucleotides include RNA and DNA, and may be isolated from natural sources, synthesized in vitro, or prepared from a combination of natural and synthetic molecules. Sizes of polynucleotides are expressed as base pairs (abbreviated “bp”), nucleotides (“nt”), or kilobases (“kb”). Where the context allows, the latter two terms may describe polynucleotides that are single-stranded or double-stranded. When the term is applied to double-stranded molecules it is used to denote overall length and will be understood to be equivalent to the term “base pairs”. It will be recognized by those skilled in the art that the two strands of a double-stranded polynucleotide may differ slightly in length and that the ends thereof may be staggered as a result of enzymatic cleavage; thus all nucleotides within a double-stranded polynucleotide molecule may not be paired.

The terms “polypeptide,” “peptide,” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, α-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refer to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

Polynucleotides encoding iron-complexed proteins can be altered for improved expression in microalgae. For example, codons in the wild-type polynucleotides encoding one or more iron-complexed proteins rarely used by the microalgae can be replaced with a codon coding for the same or a similar amino acid residue that is more commonly used by the microalgae (i.e., employing algal chloroplast codon bias), thereby allowing for more efficient expression of the iron-complexed protein and higher yields of the expressed iron-complexed protein in the microalgae, in comparison to expression of the iron-complexed protein from the wild-type polynucleotide. Methods for altering polynucleotides for improved expression in microalgae, particularly in a Chlamydomonas reinhardtii host cell, are known in the art and described in, e.g., Franklin et al., (2002) Plant J 30:733-744; Fletcher, et al., Adv Exp Med Biol. (2007) 616:90-8; Heitzer, et al., Adv Exp Med Biol. (2007) 616:46-53; Rasala and Mayfield, Bioeng Bugs. (2011) 2(1):50-4; Rasala, et al, Plant Biotechnol J. (2010) 8(6):719-33; Wu, et al., Bioresour Technol. (2011) 102(3):2610-6; Morton, J Mol Evol. (1993) 37(3):273-80; Morton, J Mol Evol. (1996) 43(1):28-31; and Morton, J Mol Evol. (1998) 46(4):449-59. Each of the foregoing references is incorporated herein by reference in its entirety.

In various embodiments, polynucleotides encoding iron-complexed proteins can be improved for expression in microalgae (e.g., algae) by changing codons that are not common in the algae host cell (e.g., used less than ˜20% of the time). A codon usage database may be found at kazusa.or.jp/codon/. For improved expression of polynucleotide sequences encoding iron-complexed protein in C. reinhardtii host cells, codons rare or not common to the chloroplast of C. reinhardtii in the nucleic acid sequences are reduced or eliminated. A representative codon table summarizing codon usage in the C. reinhardtii chloroplast is found on the internet at kazusa.or.jp/codon/cgi-bin/showcodon.cgi?species=3055.chloroplast. In various embodiments, preferred or more common codons for amino acid residues in C. reinhardtii are as follows:

| | Preferred codons |
|---|---|
| Amino Acid | for improved |
| Residue | expression in algae |
| Ala | GCT, GCA |
| Arg | CGT |
| Asn | AAT |
| Asp | GAT |
| Cys | TGT |
| Gln | CAA |
| Glu | GAA |
| Gly | GGT |
| Ile | ATT |
| His | CAT |
| Leu | TTA |
| Lys | AAA |
| Met | ATG |
| Phe | TTT |
| Pro | CCA |
| Ser | TCA |
| Thr | ACA, ACT |
| Trp | TGG |
| Tyr | TAT |
| Val | GTT, GTA |
| STOP | TAA |

In certain instances, less preferred or less common codons for expression in an algal host cell can be included in a polynucleotide sequence encoding an iron-complexed protein, for example, to avoid sequences of multiple or extended codon repeats, or sequences of reduced stability (e.g., extended A/T-rich sequences), or having a higher probability of secondary structure that could reduce or interfere with expression efficiency. In various embodiments, the polynucleotide sequence can be synthetically prepared. For example, the desired amino acid sequence of an iron-complexed protein as described herein can be entered into a software program with algorithms for determining codon usage for a photosynthetic (e.g., algal) host cell. Illustrative software includes GeneDesigner available from DNA 2.0, on the internet at dna20.com/genedesigner2.

The terms “identical” or percent “identity,” in the context of two or more nucleic acids or polypeptide sequences, refer to two or more sequences or subsequences that are the same or have a specified percentage of amino acid residues or nucleotides that are the same (i.e., share at least about 80% identity, for example, at least about 85%, 90%, 91%, 92%, 93%, 94%, 95%, 96%, 97%, 98%, 99% identity over a specified region to a reference sequence when compared and aligned for maximum correspondence over a comparison window, or designated region as measured using one of the following sequence comparison algorithms or by manual alignment and visual inspection. Such sequences are then said to be “substantially identical.” This definition also refers to the compliment of a test sequence. Preferably, the identity exists over a region that is at least about 25 amino acids or nucleotides in length, for example, over a region that is 50, 100, 200, 300, 400 amino acids or nucleotides in length, or over the full-length of a reference sequence.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins, the BLAST and BLAST 2.0 algorithms and the default parameters are used.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., eds., Current Protocols in Molecular Biology (1995 supplement)). Examples of algorithms that are suitable for determining percent sequence identity and sequence similarity are the BLAST and BLAST 2.0 algorithms, which are described in Altschul et al., J. Mol. Biol. 215:403-410 (1990) and Altschul et al., Nucleic Acids Res. 25:3389-3402 (1977), respectively. Software for performing BLAST analyses is publicly available through the National Center for Biotechnology Information (on the worldwide web at ncbi.nlm.nih.gov/).

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

In some embodiments, the nucleic acid molecule encoding myoglobin comprises a polynucleotide having at least about 60% sequence identity, e.g., at least about 65%, 70%, 75%, 80%, 85%, 90%, 95%, 98% or 99% sequence identity, to SEQ ID NO: 1. In some embodiments, the nucleic acid molecule encoding leghemoglobin comprises a polynucleotide having at least about 60% sequence identity, e.g., at least about 65%, 70%, 75%, 80%, 85%, 90%, 95%, 98% or 99% sequence identity, to SEQ ID NO: 2. Homologous sequences are, for example, those that have at least 50%, at least 60%, at least 70%, at least 80%, at least 90%, at least 95%, at least 98%, or at least 99% sequence identity to a reference amino acid sequence or nucleotide sequence, for example, the amino acid sequence or nucleotide sequence that is found in the host cell from which the protein is naturally obtained from or derived from. A nucleotide sequence can also be homologous to a codon-optimized gene sequence. For example, a nucleotide sequence can have, for example, at least 50%, at least 60%, at least 70%, at least 80%, at least 90%, at least 95%, at least 98%, or at least 99% nucleic acid sequence identity to the codon-optimized gene sequence.

Bovine Myoglobin nucleic acid sequence (SEQ ID NO: 1):
ATG GGT TTA TCA GAT GGT GAA TGG CAA TTA GTT TTA AAC GCA
TGG GGT AAA GTA GAA GCA GAT GTA GCT GGT CAT GGT CAA GAA
GTT TTA ATT CGT TTA TTT ACT GGT CAT CCT GAA ACA TTA GAA
AAA TTC GAT AAA TTC AAA CAT TTA AAA ACT GAA GCT GAA ATG
AAA GCT TCA GAA GAT TTA AAA AAA CAT GGT AAC ACA GTT TTA
ACA GCA TTA GGT GGT ATT TTA AAA AAA AAA GGT CAC CAT GAA
GCT GAA GTT AAA CAT TTA GCA GAA TCA CAC GCA AAC AAA CAT
AAA ATT CCA GTA AAA TAT TTA GAA TTT ATT TCA GAT GCT ATT
ATT CAT GTT TTA CAT GCT AAA CAC CCA TCA GAT TTT GGT GCA
GAT GCT CAA GCT GCT ATG TCA AAA GCA TTA GAA TTA TTT CGT
AAC GAT ATG GCA GCA CAA TAT AAA GTT TTA GGT TTC CAC GGT
GAC TAC AAA GAC GAC GAT GAC AAA TAA
Bovine Myoglobin amino acid sequence (SEQ ID NO: 3):
MGLSDGEWQLVLNAWGKVEADVAGHGQEVLIRLFTGHPETLEKFDKFKHLKTEAEMKASEDLKK
HGNTVLTALGGILKKKGHHEAEVKHLAESHANKHKIPVKYLEFISDAIIHVLHAKHPSDFGADA
QAAMSKALELFRNDMAAQYKVLGFHGDYKDDDDK
Leghemoglobin nucleic acid sequence (SEQ ID NO: 2):
ATG GGT GCA TTT ACA GAA AAA CAA GAA GCT TTA GTT AGT TCA
TCA TTT GAA GCT TTC AAA GCT AAC ATT CCA CAA TAT TCT GTA
GTA TTT TAT ACT TCA ATT TTA GAA AAA GCA CCA GCA GCT AAA
GAT TTA TTC AGT TTT TTA TCT AAT GGT GTT GAC CCT TCA AAT
CCA AAA TTA ACT GGT CAC GCT GAA AAA TTA TTC GGT TTA GTT
CGT GAT AGT GCA GGT CAA TTA AAA GCA AAT GGT ACA GTT GTT
GCT GAT GCT GCA TTA GGT TCT ATT CAT GCA CAA AAA GCT ATT
ACT GAT CCA CAA TTC GTA GTT GTT AAA GAA GCT CTT TTA AAA
ACA ATT AAA GAA GCA GTT GGT GAT AAA TGG TCA GAT GAA TTA
TCA TCA GCA TGG GAA GTA GCT TAT GAT GAA TTA GCT GCT GCT
ATT AAA AAA GCT TTC GAC TAC AAA GAT GAC GAC GAT AAA TAA
Leghemoglobin amino acid sequence (SEQ ID NO: 4):
MGAFTEKQEALVSSSFEAFKANIPQYSVVFYTSILEKAPAAKDLFSFLSNGVDPSNPKLTGHAE
KLFGLVRDSAGQLKANGTVVADAALGSIHAQKAITDPQFVVVKEALLKTIKEAVGDKWSDELSS
AWEVAYDELAAAIKKAFDYKDDDDK

Affinity tags can be attached to proteins so that they can be purified from their crude biological source using an affinity technique. These include, for example, chitin binding protein (CBP), maltose binding protein (MBP), and glutathione-5-transferase (GST). The poly(His) tag is a widely-used protein tag that binds to metal matrices. Some affinity tags have a dual role as a solubilization agent, such as MBP and GST. Chromatography tags are used to alter chromatographic properties of the protein to afford different resolutions across a particular separation technique. Often, these contain polyanionic amino acids, such as a FLAG-tag. Epitope tags are short peptide sequences which are chosen because high-affinity antibodies can be reliably produced in many different species. These are usually derived from viral genes, which explain their high immunoreactivity. Epitope tags include, but are not limited to, V5-tag, c-myc-tag, Strep II-tag, and hemagglutinin (HA)-tag. These tags are particularly useful for Western blotting and immunoprecipitation experiments, although they also find use in antibody purification. Fluorescence tags can be used to give a visual readout of a protein. GFP and its variants are the most commonly used fluorescence tags. More advanced applications of GFP include using it as a folding reporter (fluorescent if folded, colorless if not).

In various embodiments, the polynucleotide sequences encoding the iron-complexed protein can further encode an amino acid sequence that promotes secretion from the cell (e.g., signal peptides). Illustrative amino acid sequences that promote secretion from the cell include without limitation the secretion signal peptides from Chlamydomonas reinhardtii ars1 (MHARKMGALAVLAVACLAAVASVAHAADTK; SEQ ID NO: 5), ars2 (MGALAVFAVACLAAVASVAHAAD; SEQ ID NO: 6) and the ER insertion signal from C. reinhardtii Bip1 (MAQWKAAVLLLALACASYGFGVWAEEEKLGTVIG; SEQ ID NO: 7). In some embodiments, the one or more iron-complexed proteins comprise an amino acid sequence that promotes retention on the surface of a cell. Illustrative amino acid sequences that promote retention on the surface (e.g., plasma membrane) of a cell include without limitation a glycosylphosphatidylinositol anchor (GPI-anchor), protein fusions to full-length or domains of cell wall components including hydroxyproline-rich glycoproteins, protein fusions to full-length or domains of agglutinins, or protein fusions to full-length or domains of outer plasma membrane proteins. In varying embodiments, the polynucleotide sequences encoding the iron-complexed protein can further encode a sequence that promotes protein accumulation. Various protein accumulation amino acid sequences are known in the art. Illustrative protein accumulation amino acid sequences include, but are not limited to:

gamma zein (Zera) (SEQ ID NO: 8):
MRVLLVALALLALAASATSTHTSGGCGCQPPPPVHLPPPVHLPPPVHLPPP
VHLPPPVHLPPPVHLPPPVHVPPPVHLPPPPCHYPTQPPRPQPHPQPHPCP
CQQPHPSPCQ;
and
hydrophobin (HBN1) (SEQ ID NO: 9):
GSSNGNGNVCPPGLFSNPQCCATQVLGLIGLDCKVPSQNVYDGTDFRNVCA
KTGAQPLCCVAPVAGQALLCQTAVGA

In various embodiments, the chloroplasts of the microalgae host cells are transformed, e.g., by homologous recombination techniques, to contain and stably express one or more polynucleotides encoding one or more iron-complexed proteins, as described herein, integrated into the chloroplast genome.

Transformation of the chloroplasts of microalgae host cells can be carried out according to techniques well known to those persons skilled in the art. Examples of such techniques include without limitation electroporation, particle bombardment, cytoplasmic or nuclear microinjection, gene gun. See, e.g., FIG. 2 of WO 2012/170125, incorporated herein by reference.

### Microalgae

The iron-complexed protein can be integrated into and expressed from the chloroplast genome or the nuclear genome of a target organism. In varying embodiments, the target organism can be eukaryotic (e.g., higher plants and algae, including microalgae) or prokaryotic (e.g., cyanobacteria).

In various embodiments, the target organism for expression of the iron-complexed protein is a photosynthetic unicellular organism. In varying embodiments, the microalgae is a cyanobacterium. The iron-complexed protein can be integrated into the genome or expressed from a plasmid of cyanobacteria. In varying embodiments, the target organism is a single-celled algae such as a microalgae. Illustrative algal target organisms (host cells) of use include without limitation Chlorophyta (green algae), Rhodophyta (red algae), Xanthophyceae (yellow-green algae), Chrysophyceae (golden algae) and Phaeophyceae (brown algae).

In some embodiments, the microalga is selected from the group consisting of Chlorophyta (green algae), Rhodophyta (red algae), Stramenopiles (heterokonts), Xanthophyceae (yellow-green algae), Glaucocystophyceae (glaucocystophytes), Chlorarachniophyceae (chlorarachniophytes), Euglenida (euglenids), Haptophyceae (coccolithophorids), Chrysophyceae (golden algae), Cryptophyta (cryptomonads), Dinophyceae (dinoflagellates), Haptophyceae (coccolithophorids), B acillariophyta (diatoms), Eustigmatophyceae (eustigmatophytes), Raphidophyceae (raphidophytes), Scenedesmaceae and Phaeophyceae (brown algae).

In some embodiments, the target organism for expression of the iron-complexed protein is selected from the group consisting of Chlamydomonas reinhardtii, Dunaliella salina, Haematococcus pluvialis, Chlorella vulgaris, Acutodesmus obliquus, and Scenedesmus dimorphus.

In some embodiments, the chloroplast is a Chlorophyta (green algae) chloroplast. In some embodiments, the green alga is selected from the group consisting of Chlamydomonas, Dunaliella, Haematococcus, Chlorella, and Scenedesmaceae. In some embodiments, the Chlamydomonas is a Chlamydomonas reinhardtii. In varying embodiments, the green algae can be a Chlorophycean, a Chlamydomonas, C. reinhardtii, C. reinhardtii 137c, or a psbA deficient C. reinhardtii strain.

In varying embodiments, the iron-complexed protein is expressed in the chloroplast, nucleus, or cell of a microalgae. Illustrative and additional microalgae species of interest include without limitation, Achnanthes orientalis, Agmenellum, Amphiprora hyaline, Amphora coffeiformis, Amphora coffeiformis linea, Amphora coffeiformis punctata, Amphora coffeiformis taylori, Amphora coffeiformis tenuis, Amphora delicatissima, Amphora delicatissima capitata, Amphora sp., Anabaena, Ankistrodesmus, Ankistrodesmus falcatus, Boekelovia hooglandii, Borodinella sp., Botryococcus braunii, Botryococcus sudeticus, Carteria, Chaetoceros gracilis, Chaetoceros muelleri, Chaetoceros muelleri subsalsum, Chaetoceros sp., Chlamydomonas sp., Chlamydomonas reinhardtii, Chlorella anitrata, Chlorella Antarctica, Chlorella aureoviridis, Chlorella candida, Chlorella capsulate, Chlorella desiccate, Chlorella ellipsoidea, Chlorella emersonii, Chlorella fusca, Chlorella fusca var. vacuolata, Chlorella glucotropha, Chlorella infusionum, Chlorella infusionum var. actophila, Chlorella infusionum var. auxenophila, Chlorella kessleri, Chlorella lobophora (strain SAG 37.88), Chlorella luteoviridis, Chlorella luteoviridis var. aureoviridis, Chlorella luteoviridis var. lutescens, Chlorella miniata, Chlorella minutissima, Chlorella mutabilis, Chlorella nocturna, Chlorella parva, Chlorella photophila, Chlorella pringsheimii, Chlorella protothecoides, Chlorella protothecoides var. acidicola, Chlorella regularis, Chlorella regularis var. minima, Chlorella regularis var. umbricata, Chlorella reisiglii, Chlorella saccharophila, Chlorella saccharophila var. ellipsoidea, Chlorella salina, Chlorella simplex, Chlorella sorokiniana, Chlorella sp., Chlorella sphaerica, Chlorella stigmatophora, Chlorella vanniellii, Chlorella vulgaris, Chlorella vulgaris, Chlorella vulgaris f. tertia, Chlorella vulgaris var. autotrophica, Chlorella vulgaris var. viridis, Chlorella vulgaris var. vulgaris, Chlorella vulgaris var. vulgaris f. tertia, Chlorella vulgaris var. vulgaris f. viridis, Chlorella xanthella, Chlorella zofingiensis, Chlorella trebouxioides, Chlorella vulgaris, Chlorococcum infusionum, Chlorococcum sp., Chlorogonium, Chroomonas sp., Chrysosphaera sp., Cricosphaera sp., Crypthecodinium cohnii, Cryptomonas sp., Cyclotella cryptica, Cyclotella meneghiniana, Cyclotella sp., Dunaliella sp., Dunaliella bardawil, Dunaliella bioculata, Dunaliella granulate, Dunaliella maritime, Dunaliella minuta, Dunaliella parva, Dunaliella peircei, Dunaliella primolecta, Dunaliella salina, Dunaliella terricola, Dunaliella tertiolecta, Dunaliella viridis, Dunaliella tertiolecta, Eremosphaera viridis, Eremosphaera sp., Ellipsoidon sp., Euglena, Franceia sp., Fragilaria crotonensis, Fragilaria sp., Gleocapsa sp., Gloeothamnion sp., Hymenomonas sp., Isochrysis aff galbana, Isochrysis galbana, Lepocinclis, Micractinium, Micractinium (UTEX LB 2614), Monoraphidium minutum, Monoraphidium sp., Nannochloris sp., Nannochloropsis salina, Nannochloropsis sp., Navicula acceptata, Navicula biskanterae, Navicula pseudotenelloides, Navicula pelliculosa, Navicula saprophila, Navicula sp., Nephrochloris sp., Nephroselmis sp., Nitschia communis, Nitzschia alexandrina, Nitzschia communis, Nitzschia dissipata, Nitzschia frustulum, Nitzschia hantzschiana, Nitzschia inconspicua, Nitzschia intermedia, Nitzschia microcephala, Nitzschia pusilla, Nitzschia pusilla elliptica, Nitzschia pusilla monoensis, Nitzschia quadrangular, Nitzschia sp., Ochromonas sp., Oocystis parva, Oocystis pusilla, Oocystis sp., Oscillatoria limnetica, Oscillatoria sp., Oscillatoria subbrevis, Pascheria acidophila, Pavlova sp., Phagus, Phormidium, Platymonas sp., Pleurochrysis carterae, Pleurochrysis dentate, Pleurochrysis sp., Prototheca wickerhamii, Prototheca stagnora, Prototheca portoricensis, Prototheca moriformis, Prototheca zopfii, Pyramimonas sp., Pyrobotrys, Sarcinoid chrysophyte, Scenedesmus armatus, Schizochytrium, Spirogyra, Spirulina platensis, Stichococcus sp., Synechococcus sp., Tetraedron, Tetraselmis sp., Tetraselmis suecica, Thalassiosira weissflogii, and Viridiella fridericiana.

In some embodiments, the host is a Chlorophyta (green algae) host cell of the genus Chlamydomonas. In some embodiments, the selected host is Chlamydomonas reinhardtii, such as in Rasala and Mayfield, Bioeng Bugs. (2011) 2(1):50-4; Rasala, et al., Plant Biotechnol J. (2011) May 2, PMID 21535358; Coragliotti, et al., Mol Biotechnol. (2011) 48(1):60-75; Specht, et al., Biotechnol Lett. (2010) 32(10):1373-83; Rasala, et al., Plant Biotechnol J. (2010) 8(6):719-33; Mulo, et al., Biochim Biophys Acta. (2011) May 2, PMID:21565160; and Bonente, et al., Photosynth Res. (2011) May 6, PMID:21547493; US Pub. No. 2012/0309939; US Pub. No. 2010/0129394; and Intl. Pub. No. WO 2012/170125. All of the foregoing references are incorporated herein by reference in their entirety for all purposes.

### Culturing of Organisms

Growth methods for production of algae are known in the art. Exemplary methodology for growth of algae useful for the methods and production of compositions described herein can be found in Int'l. Pub. No. WO2018/038960, which is incorporated by reference herein in its entirety. In addition, methods for modification of such strains for production of proteins useful for use with the methods and compositions provided herein can be found in U.S. Pat. No. 9,732,351 and US Pub. Nos. 20160369291, 20170342434 and 20160257730, which are incorporated by reference herein in their entirety.

Introduction of Polynucleotide into a Host Organism or Cell

To generate a genetically modified host cell, a polynucleotide, or a polynucleotide cloned into a vector, is introduced stably or transiently into a host cell, using established techniques, including, but not limited to, electroporation, biolistic, calcium phosphate precipitation, DEAE-dextran mediated transfection, and liposome-mediated transfection. For transformation, a polynucleotide of the present disclosure will generally further include a selectable marker, e.g., any of several well-known selectable markers such as neomycin resistance, ampicillin resistance, tetracycline resistance, chloramphenicol resistance, and kanamycin resistance, zeocin resistance, hygromycin resistance and paromomycin resistance.

A polynucleotide or recombinant nucleic acid molecule described herein, can be introduced into a cell (e.g., alga cell) by a variety of methods, which are well known in the art and selected, in part, based on the particular host cell. For example, a polynucleotide can be introduced into a cell using a direct gene transfer method such as electroporation or microprojectile mediated (biolistic) transformation using a particle gun, or the “glass bead method,” or by pollen-mediated transformation, liposome-mediated transformation, transformation using wounded or enzyme-degraded immature embryos, or wounded or enzyme-degraded embryogenic callus (for example, as described in Potrykus, Ann. Rev. Plant. Physiol. Plant Mol. Biol. 42:205-225, 1991, which is incorporated herein by reference).

As discussed above, microprojectile mediated transformation can be used to introduce a polynucleotide into a cell (for example, as described in Klein et al., Nature 327:70-73, 1987, which is incorporated herein by reference). This method utilizes microprojectiles such as gold or tungsten, which are coated with the desired polynucleotide by precipitation with calcium chloride, spermidine or polyethylene glycol. The microprojectile particles are accelerated at high speed, into a cell using a device such as the BIOLISTIC PD-1000 particle gun (BioRad; Hercules Calif.). Methods for the transformation using biolistic methods are well known in the art (for example, as described in Christou, Trends in Plant Science 1:423-431, 1996).

The basic techniques used for transformation and expression in microalgae are similar to those commonly used for E. coli, Saccharomyces cerevisiae and other species. Transformation methods customized for photosynthetic microorganisms, e.g., the chloroplast of a strain of algae, are known in the art. These methods have been described in a number of texts for standard molecular biological manipulation (see Packer & Glaser, 1988, “Cyanobacteria”, Meth. Enzymol., Vol. 167; Weissbach & Weissbach, 1988, “Methods for plant molecular biology,” Academic Press, New York, Sambrook, Fritsch & Maniatis, 1989, “Molecular Cloning: A laboratory manual,” 2nd edition Cold Spring Harbor Laboratory Press, Cold Spring Harbor, N.Y.; and Clark M S, 1997, Plant Molecular Biology, Springer, N.Y.). These methods include, for example, biolistic devices (See, for example, Sanford, Trends In Biotech. (1988) .delta.: 299-302, U.S. Pat. No. 4,945,050; electroporation (Fromm et al., Proc. Nat'l. Acad. Sci. (USA) (1985) 82: 5824-5828); use of a laser beam, electroporation, microinjection or any other method capable of introducing DNA into a host cell.

Plastid transformation is a routine and well-known method for introducing a polynucleotide into a plant cell chloroplast (see U.S. Pat. Nos. 5,451,513, 5,545,817, and 5,545,818; WO 95/16783; McBride et al., Proc. Natl. Acad. Sci., USA 91:7301-7305, 1994). In some embodiments, chloroplast transformation involves introducing regions of chloroplast DNA flanking a desired nucleotide sequence, allowing for homologous recombination of the exogenous DNA into the target chloroplast genome. In some instances, one to 1.5 kb flanking nucleotide sequences of chloroplast genomic DNA may be used. Using this method, point mutations in the chloroplast 16S rRNA and rps12 genes, which confer resistance to spectinomycin and streptomycin, can be utilized as selectable markers for transformation (Svab et al., Proc. Natl. Acad. Sci., USA 87:8526-8530, 1990), and can result in stable homoplasmic transformants, at a frequency of approximately one per 100 bombardments of target leaves.

A further refinement in chloroplast transformation/expression technology that facilitates control over the timing and tissue pattern of expression of introduced DNA coding sequences in plant plastid genomes has been described in International Pub. No. WO 95/16783 and U.S. Pat. No. 5,576,198. This method involves the introduction into plant cells of constructs for nuclear transformation that provide for the expression of a viral single subunit RNA polymerase and targeting of this polymerase into the plastids via fusion to a plastid transit peptide. Transformation of plastids with DNA constructs comprising a viral single subunit RNA polymerase-specific promoter specific to the RNA polymerase expressed from the nuclear expression constructs operably linked to DNA coding sequences of interest permits control of the plastid expression constructs in a tissue and/or developmental specific manner in plants comprising both the nuclear polymerase construct and the plastid expression constructs. Expression of the nuclear RNA polymerase coding sequence can be placed under the control of either a constitutive promoter, or a tissue- or developmental stage-specific promoter, thereby extending this control to the plastid expression construct responsive to the plastid-targeted, nuclear-encoded viral RNA polymerase.

As used herein, the terms “functionally linked” and “operably linked” are used interchangeably and refer to a functional relationship between two or more DNA segments, in particular gene sequences to be expressed and those sequences controlling their expression. For example, a promoter/enhancer sequence, including any combination of cis-acting transcriptional control elements is operably linked to a coding sequence if it stimulates or modulates the transcription of the coding sequence in an appropriate host cell or other expression system. Promoter regulatory sequences that are operably linked to the transcribed gene sequence are physically contiguous to the transcribed sequence.

Of interest are transit peptide sequences derived from enzymes known to be imported into the plastids (e.g., chloroplast, leucoplast, amyloplast, etc.) of seeds. Examples of enzymes containing useful transit peptides include those related to lipid biosynthesis (e.g., subunits of the plastid-targeted dicot acetyl-CoA carboxylase, biotin carboxylase, biotin carboxyl carrier protein, a-carboxy-transferase, and plastid-targeted monocot multifunctional acetyl-CoA carboxylase (Mw, 220,000)); plastidic subunits of the fatty acid synthase complex (e.g., acyl carrier protein (ACP), malonyl-ACP synthase, KASI, KASII, and KASIII); steroyl-ACP desaturase; thioesterases (specific for short, medium, and long chain acyl ACP); plastid-targeted acyl transferases (e.g., glycerol-3-phosphate and acyl transferase); enzymes involved in the biosynthesis of aspartate family amino acids; phytoene synthase; gibberellic acid biosynthesis (e.g., ent-kaurene synthases 1 and 2); and carotenoid biosynthesis (e.g., lycopene synthase).

In some embodiments, an alga is transformed with one or more polynucleotides which encode one or more iron-complexed proteins. In various embodiments, such a transformation may introduce a nucleic acid into a plastid of the host alga (e.g., chloroplast). In other embodiments, such a transformation may introduce a nucleic acid into the nuclear genome of the host alga. In still other embodiments, such a transformation may introduce nucleic acids into both the nuclear genome and into a plastid.

Transformed cells can be plated on selective media following introduction of exogenous nucleic acids. This method may also include several steps for screening. A screen of primary transformants can be conducted to determine which clones have proper insertion of the exogenous nucleic acids. Clones which show the proper integration may be propagated and re-screened to ensure genetic stability. Such methodology ensures that the transformants contain the genes of interest. In many instances, such screening is performed by polymerase chain reaction (PCR); however, any other appropriate technique known in the art may be utilized. Many different methods of PCR are known in the art (e.g., nested PCR, real time PCR). For any given screen, one of skill in the art will recognize that PCR components may be varied to achieve optimal screening results. For example, magnesium concentration may need to be adjusted upwards when PCR is performed on disrupted alga cells. Following the screening for clones with the proper integration of exogenous nucleic acids, clones can be screened for the presence of the encoded protein(s) and/or products. Protein expression screening can be performed by Western blot analysis and/or enzyme activity assays. Transporter and/or product screening may be performed by any method known in the art, for example ATP turnover assay, substrate transport assay, HPLC or gas chromatography.

The expression of the iron-complexed protein can be accomplished by inserting a nucleic acid molecule (gene) encoding the protein into the chloroplast and/or nuclear genome of a microalgae. The modified strain of microalgae can be made homoplasmic to ensure that the polynucleotide will be stably maintained in the chloroplast genome of all descendants. A microalga is homoplasmic for a gene when the inserted gene is present in all copies of the chloroplast genome, for example. It is apparent to one of skill in the art that a chloroplast may contain multiple copies of its genome, and therefore, the term “homoplasmic” or “homoplasmy” refers to the state where all copies of a particular locus of interest are substantially identical. Plastid expression, in which genes are inserted by homologous recombination into all of the several thousand copies of the circular plastid genome present in each plant cell, takes advantage of the enormous copy number advantage over nuclear-expressed genes to permit expression levels that can readily exceed 10% or more of the total soluble plant protein. The process of determining the plasmic state of an organism of the present disclosure involves screening transformants for the presence of exogenous nucleic acids and the absence of wild-type nucleic acids at a given locus of interest.

### Vectors

The terms “construct”, “vector” and “plasmid” are used interchangeably throughout the present disclosure. Nucleic acid molecules encoding the proteins described herein can be contained in vectors, including cloning and expression vectors. A cloning vector is a self-replicating DNA molecule that serves to transfer a DNA segment into a host cell. Three common types of cloning vectors are bacterial plasmids, phages, and other viruses. An expression vector is a cloning vector designed so that a coding sequence inserted at a particular site will be transcribed and translated into a protein. Both cloning and expression vectors can contain DNA segment(s) that allow the vectors to replicate in one or more suitable host cells. In cloning vectors, this sequence is generally one that enables the vector to replicate independently of the host cell chromosomes, and also includes either origins of replication or autonomously replicating sequences.

An exogenous nucleic acid molecule encoding an iron-complexed protein can be flanked by two homologous sequences, one on each side. The first and second homologous sequences enable recombination of the exogenous sequence into the genome of the host organism to be transformed. The first and second homologous sequences can be at least 100, at least 200, at least 300, at least 400, at least 500, or at least 1500 nucleotides in length.

In some embodiments, about 0.5 to about 1.5 kb flanking nucleotide sequences of chloroplast genomic DNA may be used. In other embodiments about 0.5 to about 1.5 kb flanking nucleotide sequences of nuclear genomic DNA may be used, or about 2.0 to about 5.0 kb may be used.

A vector in some embodiments provides for amplification of the copy number of a polynucleotide. A vector can be, for example, an expression vector that provides for expression of an iron-complexed protein in an algal host cell.

A regulatory or control element, as the term is used herein, broadly refers to a nucleotide sequence that regulates the transcription or translation of a polynucleotide or the localization of a polypeptide to which it is operatively linked. Examples include, but are not limited to, a ribosome binding site (RBS), a promoter, enhancer, transcription terminator, a hairpin structure, an RNAase stability element, an initiation (start) codon, a splicing signal for intron excision and maintenance of a correct reading frame, a STOP codon, an amber or ochre codon, and an IRES. A regulatory element can include a promoter and transcriptional and translational stop signals. Elements may be provided with linkers for the purpose of introducing specific restriction sites facilitating ligation of the control sequences with the coding region of a nucleotide sequence encoding a polypeptide. Additionally, a sequence comprising a cell compartmentalization signal (i.e., a sequence that targets a polypeptide to the endoplasmic reticulum (ER), cytosol, nucleus, chloroplast membrane or cell membrane) can be attached to the polynucleotide encoding a protein of interest. Such signals are well known in the art and have been widely reported (see, e.g., U.S. Pat. No. 5,776,689).

As used herein, a “promoter” is defined as a regulatory DNA sequence generally located upstream of a gene that mediates the initiation of transcription by directing RNA polymerase to bind to DNA and initiate RNA synthesis. A “constitutive” promoter is, for example, a promoter that is active under most environmental and developmental conditions. Constitutive promoters can, for example, maintain a relatively constant level of transcription. An “inducible” promoter is a promoter that is active under controllable environmental or developmental conditions. For example, inducible promoters are promoters that initiate increased levels of transcription from DNA under their control in response to some change in the environment, e.g. the presence or absence of a nutrient or a change in temperature. Examples of inducible promoters/regulatory elements include, for example, a nitrate-inducible promoter (for example, as described in Bock et al., Plant Mol. Bio. 17:9 (1991)), or a light-inducible promoter, (for example, as described in Feinbaum et al., Mol. Gen. Genet. 226:449 (1991); and Lam and Chua, Science 248:471 (1990)), or a heat responsive promoter (for example, as described in Muller et al., Gene 111: 165-73 (1992)).

In some embodiments, a gene encoding an iron-complexed protein of the present disclosure is operably linked to an inducible promoter. Inducible promoters are well known in the art. Suitable inducible promoters include, but are not limited to, the pL of bacteriophage λ; Placo; Ptrp; Ptac (Ptrp-lac hybrid promoter); an isopropyl-beta-D-thiogalactopyranoside (IPTG)-inducible promoter, e.g., a lacZ promoter; a tetracycline-inducible promoter; an arabinose inducible promoter, e.g., P_{BAD} (for example, as described in Guzman et al. (1995) J. Bacteriol. 177:4121-4130); a xylose-inducible promoter, e.g., Pxyl (for example, as described in Kim et al. (1996) Gene 181:71-76); a GAL1 promoter; a tryptophan promoter; a lac promoter; an alcohol-inducible promoter, e.g., a methanol-inducible promoter, an ethanol-inducible promoter; a raffinose-inducible promoter; and a heat-inducible promoter, e.g., heat inducible lambda P_{L} promoter and a promoter controlled by a heat-sensitive repressor (e.g., C1857-repressed lambda-based expression vectors; for example, as described in Hoffmann et al. (1999) FEMS Microbiol Lett. 177(2):327-34).

In some embodiments, a gene encoding an iron-complexed protein of the present disclosure is operably linked to a constitutive promoter. Suitable constitutive promoters for use in prokaryotic cells are known in the art and include, but are not limited to, a sigma70 promoter, and a consensus sigma70 promoter.

A selectable marker (or selectable gene) generally is a molecule that, when present or expressed in a cell, provides a selective advantage (or disadvantage) to the cell containing the marker, for example, the ability to grow in the presence of an agent that otherwise would kill the cell. The selection gene can encode for a protein necessary for the survival or growth of the host cell transformed with the vector.

A selectable marker or selection marker can provide a means to obtain, for example, prokaryotic cells, eukaryotic cells, and/or plant cells that express the marker and, therefore, can be useful as a component of a vector of the disclosure. The selection gene or marker can encode a protein necessary for the survival or growth of the host cell transformed with the vector. One class of selectable markers are native or modified genes which restore a biological or physiological function to a host cell (e.g., restores photosynthetic capability or restores a metabolic pathway). Other examples of selectable markers include, but are not limited to, those that confer antimetabolite resistance, for example, dihydrofolate reductase, which confers resistance to methotrexate (for example, as described in Reiss, Plant Physiol. (Life Sci. Adv.) 13:143-149, 1994); neomycin phosphotransferase, which confers resistance to the aminoglycosides neomycin, kanamycin and paromycin (for example, as described in Herrera-Estrella, EMBO J. 2:987-995, 1983), hygro, which confers resistance to hygromycin (for example, as described in Marsh, Gene 32:481-485, 1984), trpB, which allows cells to utilize indole in place of tryptophan; hisD, which allows cells to utilize histinol in place of histidine (for example, as described in Hartman, Proc. Natl. Acad. Sci., USA 85:8047, 1988); mannose-6-phosphate isomerase which allows cells to utilize mannose (for example, as described in Int'l Pub. No. WO 94/20627); ornithine decarboxylase, which confers resistance to the ornithine decarboxylase inhibitor, 2-(difluoromethyl)-DL-ornithine (DFMO; for example, as described. In McConlogue, 1987, In: Current Communications in Molecular Biology, Cold Spring Harbor Laboratory ed.); and deaminase from Aspergillus terreus, which confers resistance to Blasticidin S (for example, as described in Tamura, Biosci. Biotechnol. Biochem. 59:2336-2338, 1995). Additional selectable markers include those that confer herbicide resistance, for example, phosphinothricin acetyltransferase gene, which confers resistance to phosphinothricin (for example, as described in White et al., Nucl. Acids Res. 18:1062, 1990; and Spencer et al., Theor. Appl. Genet. 79:625-631, 1990), a mutant EPSPV-synthase, which confers glyphosate resistance (for example, as described in Hinchee et al., BioTechnology 91:915-922, 1998), a mutant acetolactate synthase, which confers imidazolione or sulfonylurea resistance (for example, as described in Lee et al., EMBO J. 7:1241-1248, 1988), a mutant psbA, which confers resistance to atrazine (for example, as described in Smeda et al., Plant Physiol. 103:911-917, 1993), or a mutant protoporphyrinogen oxidase (for example, as described in U.S. Pat. No. 5,767,373), or other markers conferring resistance to an herbicide such as glufosinate. Selectable markers include polynucleotides that confer dihydrofolate reductase (DHFR) or neomycin resistance for eukaryotic cells; tetramycin or ampicillin resistance for prokaryotes such as E. coli; and bleomycin, gentamycin, glyphosate, hygromycin, kanamycin, methotrexate, phleomycin, phosphinotricin, spectinomycin, dtreptomycin, streptomycin, sulfonamide and sulfonylurea resistance in plants (for example, as described in Maliga et al., Methods in Plant Molecular Biology, Cold Spring Harbor Laboratory Press, 1995, page 39). The selection marker can have its own promoter or its expression can be driven by a promoter driving the expression of a polypeptide of interest. The promoter driving expression of the selection marker can be a constitutive or an inducible promoter.

In various embodiments the protein described herein is modified by the addition of an N-terminal or C-terminal affinity tag or epitope tag, as described herein, to aid in the detection of protein expression, and to facilitate protein purification.

In various embodiments, an iron-complexed protein described herein can be fused at the amino-terminus to the carboxy-terminus of a highly expressed protein (a fusion partner) to create a fusion protein. A fusion partner may enhance the expression level or stability of the iron-complexed protein. Engineered processing sites, for example, protease, proteolytic, or tryptic processing or cleavage sites, can be used to liberate the iron-complexed protein from the fusion partner, allowing for the purification of the desired iron-complexed protein. Examples of fusion partners that can be fused to an iron-complexed protein include, but are not limited to the mammary-associated serum amyloid (MAA) protein, the large and/or small subunit of ribulose bisphosphate carboxylase, the glutathione S-transferase (GST) gene, a thioredoxin (TRX) protein, a maltose-binding protein (MBP), one or more of the following E. coli proteins NusA, NusB, NusG, or NusE, a ubiqutin (Ub) protein, a small ubiquitin-related modifier (SUMO) protein, and a cholera toxin B subunit (CTB) protein. In various embodiments, a string of consecutive histidine residues may be linked to the 3′ end of a encoding the MBP-encoding malE gene. In various embodiments, the exogenous nucleic acid molecule may include the promoter and leader sequence of a galactokinase gene, or the leader sequence of the ampicillinase gene.

In some instances, the vectors of the present disclosure will contain elements such as an E. coli or S. cerevisiae origin of replication. Such features, combined with appropriate selectable markers, allows for the vector to be “shuttled” between the target host cell and a bacterial and/or yeast cell. The ability to passage a shuttle vector of the disclosure in a secondary host may allow for more convenient manipulation of the features of the vector. For example, a reaction mixture containing the vector and inserted polynucleotide(s) of interest can be transformed into prokaryote host cells such as E. coli, amplified and collected using routine methods, and examined to identify vectors containing an insert or construct of interest. If desired, the vector can be further manipulated, for example, by performing site-directed mutagenesis of the inserted polynucleotide, then again amplifying and selecting vectors having a mutated polynucleotide of interest. A shuttle vector then can be introduced into plant cell chloroplasts, wherein a polypeptide of interest can be expressed and, if desired, isolated according to a method of the disclosure.

Knowledge of the chloroplast or nuclear genome of the host organism, for example, C. reinhardtii, is useful in the construction of vectors for use in the disclosed embodiments. Chloroplast vectors and methods for selecting regions of a chloroplast genome for use as a vector are well known (see, for example, Bock, J. Mol. Biol. 312:425-438, 2001; Staub and Maliga, Plant Cell 4:39-45, 1992; and Kavanagh et al., Genetics 152:1111-1122, 1999, each of which is incorporated herein by reference). The entire chloroplast genome of C. reinhardtii is available to the public on the world wide web at biology.duke.edu/chlamy_genome/-chloro.html (see “view complete genome as text file” link and “maps of the chloroplast genome” link; J. Maul, J. W. Lilly, and D. B. Stern, unpublished results; revised Jan. 28, 2002; to be published as GenBank Ace. No. AF396929; and Maul, J. E., et al. (2002) The Plant Cell, Vol. 14 (2659-2679)). Generally, the nucleotide sequence of the chloroplast genomic DNA that is selected for use is not a portion of a gene, including a regulatory sequence or coding sequence. For example, the selected sequence is not a gene that if disrupted, due to the homologous recombination event, would produce a deleterious effect with respect to the chloroplast. For example, a deleterious effect on the replication of the chloroplast genome or to a plant cell containing the chloroplast. In this respect, the website containing the C. reinhardtii chloroplast genome sequence also provides maps showing coding and non-coding regions of the chloroplast genome, thus facilitating selection of a sequence useful for constructing a vector (also described in Maul, I. E., et al. (2002) The Plant Cell, Vol. 14 (2659-2679)). For example, the chloroplast vector, p322, is a clone extending from the Eco (Eco RI) site at about position 143.1 kb to the Xho (Xho I) site at about position 148.5 kb (see, world wide web at biology.duke.edu/chlamy_genome/chloro.html, and clicking on “maps of the chloroplast genome” link, and “140-150 kb” link; also accessible directly on world wide web at URL “biology.duke.edu/chlam-y/chloro/chloro140.html”).

In addition, the entire nuclear genome of C. reinhardtii is described in Merchant, S. S., et al., Science (2007), 318(5848):245-250, thus facilitating one of skill in the art to select a sequence or sequences useful for constructing a vector.

### Iron-Complexed Protein Expression

In some embodiments, the one or more iron-complexed proteins are produced in a genetically modified host cell at a level that is at least about 0.05%, at least about 0.1%, at least about 0.5%, at least about 1%, at least about 1.5%, at least about 2%, at least about 2.5%, at least about 3%, at least about 3.5%, at least about 4%, at least about 4.5%, or at least about 5% of the total soluble protein produced by the cell. In other embodiments, the iron-complexed protein is produced in a genetically modified host cell at a level that is at least about 0.01%, at least about 0.1%, or at least about 1% of the total soluble protein produced by the cell. In other embodiments, the iron-complexed protein is produced in a genetically modified host cell at a level that is at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, or at least about 70% of the total soluble protein produced by the cell.

Expression of the iron-complexed protein peptides in the microalgae host cell can be detected using any method known in the art, e.g., including immunoassays (ELISA, Western Blot) and/or nucleic acid assays (RT-PCR). Sequences of expressed polypeptides can be confirmed using any method known in the art (e.g., mass spectrometry). For example, an ELISA assay or protein mass spectrometry (for example, as described in Varghese, R. S. and Ressom, H. W., Methods Mol. Bio. (2010) 694:139-150) can also be used to determine percent total soluble protein.

Iron-complexed proteins expressed in a photosynthetic (e.g., algal) host cell are generally properly folded without performing denaturation and refolding. Furthermore, the polypeptides expressed in the chloroplast genome are not glycosylated, so coding sequences do not typically need to be altered to remove glycosylation sites and glycosylated moieties do not typically need to be removed post-translationally.

### Compositions

Further provided are compositions comprising the one or more iron-complexed proteins. Generally, the iron-complexed protein need not be purified or isolated from the host cell. Accordingly, in varying embodiments, the compositions include the microalgae host cells which have been engineered to express the one or more iron-complexed proteins. In varying embodiments, the compositions are edible by a mammal. Thus, the edible compositions can take the form of a beverage, a food, a food supplement, a nutraceutical or imitation meat.

In some aspects, an edible ingredient, composition or product includes whole microalgae biomass, such as whole biomass of an algae such as a Chlamydomonas sp. (e.g., Chlamydomonas reinhardtii). In some aspects, an edible ingredient, composition or product includes a portion of the microalgae biomass that is enriched in the iron-complexed protein as compared to the whole microalgae biomass, such as by fractionating or otherwise separating components of the microalgae biomass from a microalgae expressing one or more iron-complexed protein.

Exemplary edible products containing a whole microalgae biomass, or a portion of the microalgae biomass that is enriched in the iron-complexed protein or an iron-complexed protein made by a microalgae and then purified or extracted include a beverage, a food, a food supplement, a nutraceutical, an imitation meat and an imitation seafood. In some embodiments, the imitation meat is formed as a burger, a patty, a ground meat, a sausage, or a beef-like food product. In some aspects a whole biomass or portion thereof from a recombinant microalgae expressing one or more iron-complexed proteins is utilized as an ingredient in constructing an edible product such as an imitation meat or an imitation seafood. In some aspects, the one or more iron-complexed proteins are one or more of hemoglobin, myoglobin, leghemoglobin, beta hemoglobin, alpha hemoglobin, flavorhemoglobin, histoglobin, a neuroglobin, a protoglobin, truncated globin, or lactoferrin. In some aspects, the recombinant microalgae from which the whole microalgae biomass, or a portion of the microalgae biomass that is enriched in the iron-complexed protein or an iron-complexed protein is derived includes one or more recombinant forms of hemoglobin, myoglobin, leghemoglobin, beta hemoglobin, alpha hemoglobin, flavorhemoglobin, histoglobin, a neuroglobin, a protoglobin, truncated globin, or lactoferrin.

In some embodiments, the recombinant microalgae from which the whole microalgae biomass, or a portion of the microalgae biomass that is enriched in the iron-complexed protein is combined with one or more edible ingredients to create an edible product. In various embodiments, the edible ingredient is a protein, a carbohydrate, a sugar, a fiber, an oil, a fat, a vitamin, a mineral, a salt, a thickener, a flavoring, a coloring or any combination thereof. In various embodiments, the protein may be wheat protein, such as wheat protein, textured wheat protein, pea protein, textured pea protein, soy protein, textured soy protein, potato protein, whey protein, yeast extract, a fungus protein such as quorn, or other plant-based protein source or any combination thereof. In some embodiments, the oil or fat source may be coconut oil, canola oil, sunflower oil, safflower oil, corn oil, olive oil, avocado oil, nut oil or other plant-based oil or fat source or any combination thereof. In some embodiments, edible product is made with a starch or other carbohydrate source such as from potato, chickpea, wheat, soy, beans, corn or other plant-based starch or carbohydrate or any combination thereof. In some embodiments, edible product is made with a thickener, for example, starches such as arrowroot, cornstarch, katakuri starch, potato starch, sago, tapioca and their starch derivatives may be used as a thickener; microbial and vegetable gums used as food thickeners include alginin, guar gum, locust bean gum, konjac and xanthan gum; and proteins such as collagen and egg whites may be used as thickeners; and sugar polymers for use as thickeners include agar, methylcellulose, carboxymethyl cellulose, pectin and carrageenan. In some embodiments, edible product is made with with vitamins and minerals in an edible composition, such as vitamin E, vitamin C, thiamine (vitamin B1), zinc, niacin, vitamin B6, riboflavin (vitamin B2), and vitamin B12.

Additional Sequence Information
Hemoglobin (Bovine) amino acid sequence (SEQ ID NO:
10):
MLTAEEKAAVTAFWGKVKVDEVGGEALGRLLVVYPWTQRFFESFGDLSTAD
AVMNNPKVKAHGKKVLDSFSNGMKHLDDLKGTFAALSELHCDKLHVDPENF
KLLGNVLVVVLARNFGKEFTPVLQADFQKVVAGVANALAHRYH
Alpha-Hemoglobin (Bovine) amino acid sequence (SEQ
ID NO: 11):
MVLSAADKGNVKAAWGKVGGHAAEYGAEALERMFLSFPTTKTYFPHFDLSH
GSAQVKGHGAKVAAALTKAVEHLDDLPGALSELSDLHAHKLRVDPVNFKLL
SHSLLVTLASHLPSDFTPAVHASLDKFLANVSTVLTSKYR
Beta-Hemoglobin (Bovine) amino acid sequence (SEQ
ID NO: 12):
MLTAEEKAAVTAFWGKVKVDEVGGEALGRLLVVYPWTQRFFESFGDLSTAD
AVMNNPKVKAHGKKVLDSFSNGMKHLDDLKGTFAALSELHCDKLHVDPENF
KLLGNVLVVVLARNFGKEFTPVLQADFQKVVAGVANALAHRYH
Myoglobin (Bovine) amino acid sequence (SEQ ID NO:
13):
MGLSDGEWQLVLNAWGKVEADVAGHGQEVLIRLFTGHPETLEKFDKFKHLK
TEAEMKASEDLKKHGNTVLTALGGILKKKGHHEAEVKHLAESHANKHKIPV
KYLEFISDAIIHVLHAKHPSDFGADAQAAMSKALELFRNDMAAQYKVLGFH
G
Leghomoglobin (Soy) amino acid sequence (SEQ ID NO:
14):
MGAFTEKQEALVSSSFEAFKANIPQYSVVFYTSILEKAPAAKDLFSFLSNG
VDPSNPKLTGHAEKLFGLVRDSAGQLKANGTVVADAALGSIHAQKAITDPQ
FVVVKEALLKTIKEAVGDKWSDELSSAWEVAYDELAAAIKKAF
Histoglobin (Human) amino acid sequence (SEQ ID NO:
15):
MEKVPGEMEIERRERSEELSEAERKAVQAMWARLYANCEDVGVAILVRFFV
NFPSAKQYFSQFKHMEDPLEMERSPQLRKHACRVMGALNTVVENLHDPDKV
SSVLALVGKAHALKHKVEPVYFKILSGVILEVVAEEFASDFPPETQRAWAK
LRGLIYSHVTAAYKEVGWVQQVPNATTPPATLPSSGP
Neuroglobin (Bovine) amino acid sequence (SEQ ID
NO: 16):
MELPEPELIRQSWREVSRSPLEHGTVLFARLFDLEPDLLPLFQYNCRQFSS
PEDCLSSPEFLDHIRKVMLVIDAAVTNVEDLSSLEEYLAGLGRKHRAVGVK
LSSFSTVGESLLYMLEKCLGPAFTPATRAAWSQLYGAVVQAMSRGWGGE
Protoglobin (Aspergillus) amino acid sequence (SEQ
ID NO: 17):
MEGLYFDSSRPIKHVDRKAIYTRLEARINYLQDFLDFNSADVEALTTGSKY
IKALIPAVVNIVYKKLLEQDITARAFHTRDTSDERPIEEFYNEESPQIMRR
KMFLRWYLTKLCSDPTQTEFWRYLNKVGMMHAAQERMHPLNIEYIHMGACL
GFIQDIFTEALMSHPRLQLQRKVALVRAIGKIIWIQNDLIAKWRIRDGEEY
AEEMSQMTLDEREGFLGDKKILGDSSSTSASSSDDDRSSVHSNPSIAPSIA
PSTISACPFADMVMSNSAASTSETKIWAGK
Truncated Globin (Rubinisphaera) amino acid
sequence (SEQ ID NO: 18):
MNSPTVYEQIGGETSVRRLVDRFYDLMSELPETSTILALHPEDLTESRNKL
FKFLSGFFGGPSLYIQEYGHPMLRARHLPFPIGESERDQWLLCMNRAIDEQ
INDPLLASELKMTFFRTADHMRNRPG
Lactoferrin (Bovine) amino acid sequence (SEQ ID
NO: 19):
MKLFVPALLSLGALGLCLAAPRKNVRWCTISQPEWFKCRRWQWRMKKLGAP
SITCVRRAFALECIRAIAEKKADAVTLDGGMVFEAGRDPYKLRPVAAEIYG
TKESPQTHYYAVAVVKKGSNFQLDQLQGRKSCHTGLGRSAGWIIPMGILRP
YLSWTESEPLQGAVAKFFSASCVPCIDRQAYPNLCQLCKGEGENQCACSSR
EPYFGYSGAFKCLQDGAGDVAFVKETTVFENLPEKADRDQYELLCLNNSRA
PVDAFKECHLAQVPSHAVVARSVDGKEDLIWKLLSKAQEKFGKNKSRSFQL
FGSPPGQRDLLFKDSALGFLRIPSKVDSALYLGSRYLTTLKNLRETAEEVK
ARYTRVVWCAVGPEEQKKCQQWSQQSGQNVTCATASTTDDCIVLVLKGEAD
ALNLDGGYIYTAGKCGLVPVLAENRKSSKHSSLDCVLRPTEGYLAVAVVKK
ANEGLTWNSLKDKKSCHTAVDRTAGWNIPMGLIVNQTGSCAFDEFFSQSCA
PGADPKSRLCALCAGDDQGLDKCVPNSKEKYYGYTGAFRCLAEDVGDVAFV
KNDTVWENTNGESTADWAKNLNREDFRLLCLDGTRKPVTEAQSCHLAVAPN
HAVVSRSDRAAHVKQVLLHQQALFGKNGKNCPDKFCLFKSETKNLLFNDNT
ECLAKLGGRPTYEEYLGTEYVTAIANLKKCSTSPLLEACAFLTR

Exemplary embodiments

Among the provided embodiments are:

Embodiment 1: A microalgae comprising a nucleic acid molecule encoding a heterologous iron-complexed protein, wherein the microalgae is capable of accumulating the iron-complexed protein to at least about 0.1% of the algae biomass. Embodiment 2: The microalgae of embodiment 1, wherein the nucleic acid further comprises a heterologous promoter. Embodiment 3: The microalgae of embodiment 1 or 2, wherein the nucleic acid is integrated into the nuclear genome of the microalgae. Embodiment 4: The microalgae according to any of embodiments 1-3, wherein the nucleic acid is integrated into the chloroplast genome of the microalgae. Embodiment 5: The microalgae according to any of embodiments 1-3, wherein the nucleic acid is integrated into the mitochondrial genome of the microalgae. Embodiment 6: The microalgae according to any of embodiments 1-5, wherein iron-complexed protein is selected from hemoglobin, myoglobin, leghemoglobin, beta hemoglobin, alpha hemoglobin, flavohemoglobin, histoglobin, a neuroglobin, a protoglobin, truncated globin, and lactoferrin.

Embodiment 7: The microalgae according to any of embodiments 1-6, wherein iron-complexed protein comprises an amino acid sequence of any of SEQ ID NOs: 3 or 4, or a sequence with at least 80% homology thereto. Embodiment 8: The microalgae according to any of embodiments 1-7, wherein the nucleic acid is codon-optimized for expression in the microalgae. Embodiment 9: The microalgae according to any of embodiments 1-8, wherein the microalgae further comprises one or more additional nucleic acids encoding a protein that enhances accumulation or stability of the iron-complexed protein. Embodiment 10: A food stuff comprising the microalgae according to any of embodiments 1-9. Embodiment 11: A nutritional supplement comprising the microalgae according to any of embodiments 1-9. Embodiment 12: A plant-based meat substitute comprising the microalgae according to any of claims 1-9.

Embodiment 13: A method for producing a recombinant iron-complexed protein in algae, comprising: integrating a nucleic acid molecule encoding an iron-complexed protein into a microalgal genome; growing the microalgae under conditions sufficient to express the iron-complexed protein and produce a microalgae biomass; and harvesting the microalgae biomass. Embodiment 14: The method of embodiment 13, wherein the iron-complexed protein is at least 0.1% of the harvested algae biomass. Embodiment 15: The method of embodiment 13, wherein the nucleic acid is integrated into the microalgal nuclear genome. Embodiment 16: The method of embodiment 13, wherein the nucleic acid is integrated into the microalgal chloroplast genome. Embodiment 17: The method of embodiment 13, wherein the nucleic acid is integrated into the microalgal mitochondrial genome.

Embodiment 18: The method of embodiment 13, wherein the iron-complexed protein accumulates in chloroplasts of the microalgae. Embodiment 19: The method of embodiment 13, wherein the iron-complexed protein accumulates in endoplasmic reticulum of the microalgae. Embodiment 20: The method of embodiment 13, wherein the iron-complexed protein accumulates in nuclear envelope of the microalgae. Embodiment 21: The method of embodiment 13, wherein the iron-complexed protein accumulates in periplasmic space of the microalgae. Embodiment 22: The method according to any of embodiments 13-21, wherein the nucleic acid is codon optimized for expression in the microalgae. Embodiment 23: The method according to any of embodiments 13-22, wherein the iron-complexed protein is hemoglobin, myoglobin, leghemoglobin, beta hemoglobin, alpha hemoglobin, flavohemoglobin, histoglobin, a neuroglobin, a protoglobin, truncated globin, or lactoferrin.

Embodiment 24: The method according to any of embodiments 13-22, wherein the iron-complexed protein comprises an amino acid sequence of any of SEQ ID NOs: 3 or 4, or a sequence with at least 80% homology thereto. Embodiment 25: The method according to any of embodiments 13-24, further comprising integrating into the microalgal genome one or more additional nucleic acids encoding a protein to enhance accumulation or stability of the iron-complexed protein.

Embodiment 26: The method according to any of embodiments 13-24, further comprising growing the microalgae heterotrophically or mixotrophically using a reduced carbon source. Embodiment 27: The method of embodiment 26, wherein the microalgae is cultivated in a bioreactor which is supplied with air or another gas mixture rich in oxygen. Embodiment 28: The method according to any of embodiments 13-27, wherein the microalgae density reaches at least 100 g/l. Embodiment 29: The method according to any of embodiments 13-28, wherein the microalgae density reaches at least about 100 g/l in less than 7 days. Embodiment 30: The method according to any of embodiments 13-29, wherein dissolved oxygen content in the bioreactor is allowed to drop below 5% once the iron-carrying protein concentration exceeds 0.1% of the algal biomass. Embodiment 31: The method according to any of embodiments 13-30, wherein the microalgae is grown under aerobic fermentation conditions. Embodiment 32: The method according to any of embodiments 13-31, wherein the microalgae is Generally Regarded As Safe (GRAS) for human consumption. Embodiment 33: The method according to any of embodiments 13-21, wherein the microalgae is a Chlamydomonas sp.

Embodiment 34: The method according to any of embodiments 13-21, wherein the microalgae is a Chlamydomonas reinhardtii. Embodiment 35: The method according to any of embodiments 13-34, wherein the microalgae used to express the iron-complexed protein turns yellow in the dark. Embodiment 36: The method according to any of embodiments 13-34, wherein the microalgae used to express the iron-complexed protein turns white in the dark. Embodiment 37: The method according to any of embodiments 13-34, wherein the microalgae strain is one with a reduced chlorophyll content. Embodiment 38: The method according to any of embodiments 13-34, wherein the microalgae accumulates heme co-factors at least 0.1% of biomass. Embodiment 39: The method of embodiment 26, wherein the reduced carbon source is a sugar. Embodiment 40: The method of embodiment 39, wherein the sugar is selected from the group consisting of fructose, sucrose, glucose, galactose, and maltose. Embodiment 41: The method of embodiment 26, wherein the reduced carbon source is not an alcohol. Embodiment 42: The method of embodiment 26, wherein the reduced carbon source is an acetate or an organic acid.

Embodiment 43: The method according to any of embodiments 13-42, further comprising separating the iron-complexed protein from the microalgae biomass. Embodiment 44: The method according to any of embodiments 13-42, further comprising incorporating the produced iron-complexed protein into a foodstuff or nutritional supplement. Embodiment 45: The method of embodiment 43, wherein the microalgae biomass comprising the iron-complexed protein is incorporated into the foodstuff or nutritional supplement. Embodiment 46: The method of embodiment 45, wherein the iron-complexed protein is isolated from the microalgae biomass prior to incorporating the iron-complexed protein into the foodstuff or nutritional supplement.

Embodiment 47: A composition comprising a microalgae biomass, wherein the biomass comprises at least about 0.1% of an iron-complexed protein. Embodiment 48: The composition of embodiment 47, wherein the iron-complexed protein is heterologous to the microalgae. Embodiment 49: The composition of embodiments 47 or 48, wherein the iron-complexed protein is selected from hemoglobin, myoglobin, leghemoglobin, beta hemoglobin, alpha hemoglobin, flavohemoglobin, histoglobin, a neuroglobin, a protoglobin, truncated globin, and lactoferrin. Embodiment 50: The composition according to any of embodiments 47-49, wherein the microalgae is a Chlamydomonas Sp. Embodiment 51: The composition according to any of embodiments 47-49, wherein the microalgae is Chlamydomonas reinhardtii. Embodiment 52: The method according to any of embodiments 13-46, wherein the algae is grown in the dark.

### EXAMPLES

In one example, the chloroplast genome of two Chlamydomonas strains, THN1 and THN6, were transformed with a bovine myoglobin gene and placed under the transcriptional control of the 16S promoter and the translational control of the psbM 5′UTR. Once homoplasmic, the strains of Chlamydomonas reinhardtii containing the bovine myoglobin gene were grown in a flask in the dark and light. Total soluble protein was isolated from each strain, THN1+myoglobin light, THN1+myoglobin dark, THN6+myoglobin Light, THN6+myoglobin dark and 20 ug separated by polyacrylamide gel electrophoresis. The protein from each sample was subsequently transferred to a nitrocellulose membrane and probed with an anti-myoglobin antibody to detect the presence of bovine myoglobin. THN6+myoglobin grown in the dark is shown to accumulate myoglobin.

What is claimed is:

1. A recombinant microalgae comprising a nucleic acid molecule encoding a heterologous iron-complexed protein, wherein the recombinant microalgae accumulates the iron-complexed protein to at least about 0.1% of the algae biomass.

2. The recombinant microalgae of claim 1, wherein the microalgae is a Chlamydomonas Sp.

3. The recombinant microalgae of claim 2, wherein the microalgae is Chlamydomonas reinhardtii.

4. The recombinant microalgae of claim 1, wherein the heterologous iron-complexed protein is selected from hemoglobin, myoglobin, leghemoglobin, beta hemoglobin, alpha hemoglobin, flavorhemoglobin, histoglobin, a neuroglobin, a protoglobin, truncated globin, and lactoferrin.

5. A composition comprising a microalgae biomass or a portion thereof of the recombinant microalgae of claim 1.

6. The composition of claim 5, wherein the heterologous iron-complexed protein is selected from hemoglobin, myoglobin, leghemoglobin, beta hemoglobin, alpha hemoglobin, flavorhemoglobin, histoglobin, a neuroglobin, a protoglobin, truncated globin, and lactoferrin.

7. The composition of claim 5, wherein the composition comprises the whole microalgae biomass.

8. An edible product comprising the composition of claim 7.

9. The composition of claim 5, wherein the composition comprises a portion of the microalgae biomass that is enriched in the heterologous iron-complexed protein as compared to the whole microalgae biomass.

10. The edible product of claim 8, wherein the edible product is selected from the group consisting of a beverage, a food, a food supplement, a nutraceutical, an imitation meat and an imitation seafood.

11. A method of making an edible product, comprising:
a) obtaining a biomass from the recombinant microalgae according to claim 1;
b) combining the biomass or a portion thereof containing the heterologous iron-complexed protein with at least one edible ingredient to create an edible product.

12. The method of claim 11, wherein the edible product is selected from the group consisting of a beverage, a food, a food supplement, a nutraceutical, an imitation meat and an imitation seafood.

13. The method of claim 11, wherein the biomass or the portion thereof is enriched for the heterologous iron-complexed protein prior to step b).

14. A method for producing a recombinant iron-complexed protein in algae, comprising:
a) integrating a nucleic acid molecule encoding an iron-complexed protein into a microalgae genome;
b) growing the microalgae under conditions sufficient to express the iron-complexed protein; and
c) harvesting the microalgae to recover the produced iron-complexed protein.

15. The method of claim 14, wherein the iron-complexed protein is hemoglobin, myoglobin, leghemoglobin, beta hemoglobin, alpha hemoglobin, flavorhemoglobin, histoglobin, a neuroglobin, a protoglobin, truncated globin, or lactoferrin.

16. The method of claim 14, further comprising growing the microalgae heterotrophically or mixotrophically using a reduced carbon source selected from the group consisting of fructose, sucrose, glucose, galactose, maltose, acetate, citric acid and acetic acid.

17. The method of claim 14, wherein the microalgae is a Chlamydomonas sp.

18. The method of claim 14, wherein the microalgae has a reduced chlorophyll content.

19. The method of claim 14, further comprising incorporating the produced iron-complexed protein into an edible product, wherein the edible product is selected from the group consisting of a beverage, a food, a food supplement, a nutraceutical, an imitation meat and an imitation seafood.

20. The method of claim 19, further comprising separating the iron-complexed protein from the microalgae biomass prior to incorporating the produced iron-complexed protein into the edible product.


SEQUENCE LISTING (19 sequences)

SEQ ID NO: 1 - DNA, 489 bp, Bos taurus
atgggtttatcagatggtgaatggcaattagttttaaacgcatggggtaaagtagaagcagatgtagctggtcatggtcaagaagttttaattcgtttatttactggtcatcctgaaacattagaaaaattcgataaattcaaacatttaaaaactgaagctgaaatgaaagcttcagaagatttaaaaaaacatggtaacacagttttaacagcattaggtggtattttaaaaaaaaaaggtcaccatgaagctgaagttaaacatttagcagaatcacacgcaaacaaacataaaattccagtaaaatatttagaatttatttcagatgctattattcatgttttacatgctaaacacccatcagattttggtgcagatgctcaagctgctatgtcaaaagcattagaattatttcgtaacgatatggcagcacaatataaagttttaggtttccacggtgactacaaagacgacgatgacaaataa

SEQ ID NO: 2 - DNA, 462 bp, Glycine soja
atgggtgcatttacagaaaaacaagaagctttagttagttcatcatttgaagctttcaaagctaacattccacaatattctgtagtattttatacttcaattttagaaaaagcaccagcagctaaagatttattcagttttttatctaatggtgttgacccttcaaatccaaaattaactggtcacgctgaaaaattattcggtttagttcgtgatagtgcaggtcaattaaaagcaaatggtacagttgttgctgatgctgcattaggttctattcatgcacaaaaagctattactgatccacaattcgtagttgttaaagaagctcttttaaaaacaattaaagaagcagttggtgataaatggtcagatgaattatcatcagcatgggaagtagcttatgatgaattagctgctgctattaaaaaagctttcgactacaaagatgacgacgataaataa

SEQ ID NO: 3 - protein, 162 aa, Bos taurus
MGLSDGEWQLVLNAWGKVEADVAGHGQEVLIRLFTGHPETLEKFDKFKHLKTEAEMKASEDLKKHGNTVLTALGGILKKKGHHEAEVKHLAESHANKHKIPVKYLEFISDAIIHVLHAKHPSDFGADAQAAMSKALELFRNDMAAQYKVLGFHGDYKDDDDK

SEQ ID NO: 4 - protein, 153 aa, Glycine max
MGAFTEKQEALVSSSFEAFKANIPQYSVVFYTSILEKAPAAKDLFSFLSNGVDPSNPKLTGHAEKLFGLVRDSAGQLKANGTVVADAALGSIHAQKAITDPQFVVVKEALLKTIKEAVGDKWSDELSSAWEVAYDELAAAIKKAFDYKDDDDK

SEQ ID NO: 5 - protein, 30 aa, Chlamydomonas reinhardtii
MHARKMGALAVLAVACLAAVASVAHAADTK

SEQ ID NO: 6 - protein, 23 aa, Chlamydomonas reinhardtii
MGALAVFAVACLAAVASVAHAAD

SEQ ID NO: 7 - protein, 34 aa, Chlamydomonas reinhardtii
MAQWKAAVLLLALACASYGFGVWAEEEKLGTVIG

SEQ ID NO: 8 - protein, 112 aa, Zea mays
MRVLLVALALLALAASATSTHTSGGCGCQPPPPVHLPPPVHLPPPVHLPPPVHLPPPVHLPPPVHLPPPVHVPPPVHLPPPPCHYPTQPPRPQPHPQPHPCPCQQPHPSPCQ

SEQ ID NO: 9 - protein, 77 aa, Trichoderma reesei
GSSNGNGNVCPPGLFSNPQCCATQVLGLIGLDCKVPSQNVYDGTDFRNVCAKTGAQPLCCVAPVAGQALLCQTAVGA

SEQ ID NO: 10 - protein, 145 aa, Bos taurus
MLTAEEKAAVTAFWGKVKVDEVGGEALGRLLVVYPWTQRFFESFGDLSTADAVMNNPKVKAHGKKVLDSFSNGMKHLDDLKGTFAALSELHCDKLHVDPENFKLLGNVLVVVLARNFGKEFTPVLQADFQKVVAGVANALAHRYH

SEQ ID NO: 11 - protein, 142 aa, Bos taurus
MVLSAADKGNVKAAWGKVGGHAAEYGAEALERMFLSFPTTKTYFPHFDLSHGSAQVKGHGAKVAAALTKAVEHLDDLPGALSELSDLHAHKLRVDPVNFKLLSHSLLVTLASHLPSDFTPAVHASLDKFLANVSTVLTSKYR

SEQ ID NO: 12 - protein, 145 aa, Bos taurus; identical to SEQ ID NO: 10

SEQ ID NO: 13 - protein, 154 aa, Bos taurus
MGLSDGEWQLVLNAWGKVEADVAGHGQEVLIRLFTGHPETLEKFDKFKHLKTEAEMKASEDLKKHGNTVLTALGGILKKKGHHEAEVKHLAESHANKHKIPVKYLEFISDAIIHVLHAKHPSDFGADAQAAMSKALELFRNDMAAQYKVLGFHG

SEQ ID NO: 14 - protein, 145 aa, Glycine max
MGAFTEKQEALVSSSFEAFKANIPQYSVVFYTSILEKAPAAKDLFSFLSNGVDPSNPKLTGHAEKLFGLVRDSAGQLKANGTVVADAALGSIHAQKAITDPQFVVVKEALLKTIKEAVGDKWSDELSSAWEVAYDELAAAIKKAF

SEQ ID NO: 15 - protein, 190 aa, Homo sapiens
MEKVPGEMEIERRERSEELSEAERKAVQAMWARLYANCEDVGVAILVRFFVNFPSAKQYFSQFKHMEDPLEMERSPQLRKHACRVMGALNTVVENLHDPDKVSSVLALVGKAHALKHKVEPVYFKILSGVILEVVAEEFASDFPPETQRAWAKLRGLIYSHVTAAYKEVGWVQQVPNATTPPATLPSSGP

SEQ ID NO: 16 - protein, 151 aa, Bos taurus
MELPEPELIRQSWREVSRSPLEHGTVLFARLFDLEPDLLPLFQYNCRQFSSPEDCLSSPEFLDHIRKVMLVIDAAVTNVEDLSSLEEYLAGLGRKHRAVGVKLSSFSTVGESLLYMLEKCLGPAFTPATRAAWSQLYGAVVQAMSRGWGGE

SEQ ID NO: 17 - protein, 285 aa, Aspergillus niger
MEGLYFDSSRPIKHVDRKAIYTRLEARINYLQDFLDFNSADVEALTTGSKYIKALIPAVVNIVYKKLLEQDITARAFHTRDTSDERPIEEFYNEESPQIMRRKMFLRWYLTKLCSDPTQTEFWRYLNKVGMMHAAQERMHPLNIEYIHMGACLGFIQDIFTEALMSHPRLQLQRKVALVRAIGKIIWIQNDLIAKWRIRDGEEYAEEMSQMTLDEREGFLGDKKILGDSSSTSASSSDDDRSSVHSNPSIAPSIAPSTISACPFADMVMSNSAASTSETKIWAGK

SEQ ID NO: 18 - protein, 128 aa, Rubinisphaera brasiliensis
MNSPTVYEQIGGETSVRRLVDRFYDLMSELPETSTILALHPEDLTESRNKLFKFLSGFFGGPSLYIQEYGHPMLRARHLPFPIGESERDQWLLCMNRAIDEQINDPLLASELKMTFFRTADHMRNRPG

SEQ ID NO: 19 - protein, 708 aa, Bos taurus
MKLFVPALLSLGALGLCLAAPRKNVRWCTISQPEWFKCRRWQWRMKKLGAPSITCVRRAFALECIRAIAEKKADAVTLDGGMVFEAGRDPYKLRPVAAEIYGTKESPQTHYYAVAVVKKGSNFQLDQLQGRKSCHTGLGRSAGWIIPMGILRPYLSWTESLEPLQGAVAKFFSASCVPCIDRQAYPNLCQLCKGEGENQCACSSREPYFGYSGAFKCLQDGAGDVAFVKETTVFENLPEKADRDQYELLCLNNSRAPVDAFKECHLAQVPSHAVVARSVDGKEDLIWKLLSKAQEKFGKNKSRSFQLFGSPPGQRDLLFKDSALGFLRIPSKVDSALYLGSRYLTTLKNLRETAEEVKARYTRVVWCAVGPEEQKKCQQWSQQSGQNVTCATASTTDDCIVLVLKGEADALNLDGGYIYTAGKCGLVPVLAENRKSSKHSSLDCVLRPTEGYLAVAVVKKANEGLTWNSLKDKKSCHTAVDRTAGWNIPMGLIVNQTGSCAFDEFFSQSCAPGADPKSRLCALCAGDDQGLDKCVPNSKEKYYGYTGAFRCLAEDVGDVAFVKNDTVWENTNGESTADWAKNLNREDFRLLCLDGTRKPVTEAQSCHLAVAPNHAVVSRSDRAAHVKQVLLHQQALFGKNGKNCPDKFCLFKSETKNLLFNDNTECLAKLGGRPTYEEYLGTEYVTAIANLKKCSTSPLLEACAFLTR